(19) United States
(12) Patent Application Publication
(10) Pub. No.: US 2018/0055669 A1
(43) Pub. Date: Mar. 1, 2018

(54) LUMINAL IMPLANT FOR THE CORRECTION OF OCCLUSIONS

(71) Applicant: Veinovo, LLC, Clayton, CA (US)

(72) Inventors: Huseyin Fertac BILGE, Dublin, CA (US); Xiao-Yan GONG, Laguna Niguel, CA (US); Michael WILLIAMSON, Clayton, CA (US)

(21) Appl. No.: 15/692,880

(22) Filed: Aug. 31, 2017

Related U.S. Application Data
(60) Provisional application No. 62/382,456, filed on Sep. 1, 2016.

(51) Int. Cl. A61F 2/915

(52) U.S. Cl. CPC A61F 2/915; A61F 2210/008; A61F 2210/0014; A61F 2002/91583

(57) ABSTRACT

Apparatus, systems, and methods for a medical implant with improved durability and strength. An implant structure having a mesh, wherein struts of the mesh vary in width as a function of length, where one or more segments are defined by one or more corresponding equations that define strut width. Connecting elements may be optionally included and may further optionally vary in width and shape. Strut tapering functions may be one or more segments of piecewise linear equations. Implant mesh structures may be symmetric, asymmetric, or may vary in segments along the length and/or circumference of the mesh. The mesh may be of a self-expanding or balloon expandable material suitable for medical use.

### CROSS REFERENCE

This application claims the benefit of U.S. Provisional Application No. 62/382,456, filed Sep. 1, 2016, which is incorporated herein by reference in its entirety.

### BACKGROUND

Venous thromboembolic disease is a common medical problem with first episode incidence ranging from 60 to 180 per 100,000 persons per year. Symptomatic lower-extremity deep vein thrombosis (DVT) has been estimated to affect more than 250,000 patients per year in the United States. The natural history and time course of DVT episodes are variable. Anticoagulation therapy halts the progression of thrombosis and is generally effective in reducing the risk of pulmonary embolism and alleviating acute symptoms such as leg swelling and pain. However, its effects on subsequent development of delayed postthrombotic chronic venous disease are questionable. A definite relationship exists between early recanalization of the thrombosed veinous segment and subsequent preservation of venous valve competence.

Postthrombotic syndrome (PTS) refers to a spectrum of postthrombotic chronic venous diseases attributable to venous hypertension and stasis that affect a limb previously involved in DVT. The spectrum of PTS can encompass several combinations of symptoms in various degrees of severity. These include chronic leg heaviness, leg aching and venous claudication, edema, venous varicosities and chronic trophic skin changes (ranging from hyperpigmentation to non-healing ulceration to fibrotic scarring). Patients who have had DVT are at risk of developing PTS; 50% of patients develop PTS within 2 years (range 35%-70%). Most patients are young (average age is 43 years) and the clinical sequalae of PTS can take a stiff toll on one's ability to perform daily functions and quality of life. The main cause of PTS is believed to be venous obstruction. Persistent venous obstruction may be more significant than venous reflux due to damage to venous valves in causing postthombotic syndrome.

PTS is thought to result from permanent functional impairment of the venous system due to residual clot after DVT. While removing residual thrombus is important, it may not be recognized as an issue by a physician until a period of time when symptoms persist. The pain and swelling caused by acute DVT may persist for months, making it challenging to diagnose PTS earlier than 3-6 months after acute DVT. More severe post thrombotic manifestations that occur after diagnosis of DVT, and more extensive thrombosis may predict worse outcome over time. At this point, residual thrombus is no longer a fresh, acute, removable clot. Currently, no endovascular treatment modality exists that removes chronic thrombus. Angioplasty techniques alone do not result in acceptable clinical outcomes. Better outcomes may be obtained by combining angioplasty and stenting. However, there are no stents on the market specifically developed to meet the requirements of the venous system, and the use of stents has therefore been very limited. Most patients are managed with anti-coagulation medications and externally applied limb compression.

The approximate US incidence of chronic thrombus leading to venous hypertension and PTS is estimated at over 200,000 annually, with having a total cost of about $ 1.2B to $2.4B annually. The clinical ability to open venous obstruction will reduce post-thrombotic morbidity and the associated economic burden.

The venous system presents both similarities and marked differences in comparison to the arterial system from a stent design perspective. For example, in designing a stent to treat venous insufficiency related to DVT, the bending, axial elongation and foreshortening, and twisting experienced by a venous stent would be expected to be similar to that experienced by an arterial stent in the vicinity of the knee and thigh. In contrast, the pulsatile deflection of a venous stent would be less than that of an arterial stent.

In further contrast, as compared to arterial thrombosis where blood vessel wall damage is required for thrombosis formation, the majority of venous thrombi form without any injured epithelium. Red blood cells and fibrin form the main components of venous thrombus, which, in turn, attach to the endothelium, normally a non-thrombogenic surface. Platelets in a venous thrombus attach to downstream fibrin, while in an arterial thrombus platelets compose the core of the thrombus. As a whole, platelets constitute proportionately less of a venous thrombus as compared to arterial thrombus, meaning that fibrin is the proportionately greater constituent of a venous thrombus thereby making venous thrombus a more tenacious form of thrombus from an interventional point of view; hence an arterial stent design does not immediately translate for successful application in the venous system.

The beginning of the venous thrombus formation process is thought to be initiated by tissue factor affected thrombin production, which leads to fibrin deposition. A clot in its acute form (usually within 17 days) can be lysed with thrombolytics or can be easily aspirated. Over time, thrombus organization begins with the infiltration of inflammatory cells into the clot. At this point the thrombus can no longer be lysed with lytics or can be aspirated because of its fibrous adherent nature. Over the following one to three months there is a fibroelastic intimal thickening at the site of thrombus attachment in most patients and the thrombus is replaced by a collagenous scar tissue devoid of all blood elements, resulting in a rubbery consistency similar to scar tissue formed on skin. Even though some recanallization occurs, what is left behind is a scarred, drastically narrowed, non-compliant vein with minimal blood flow. This affected tissue requires comparatively higher radial force to restore patency, while also being located in the venous system whose biomechanics have important differences from the arterial system, meaning that a unique combination of design factors may determine an optimal solution to the venous thrombus problem.

Because of the increased need for radial stiffness to hold patent the tissue of a chronic venous occlusion, there remains a need for a stent design having comparatively high radial stiffness while preserving flexibility and fatigue resistance.

### SUMMARY

Described herein is a stent that operates in biologic and physiologic harmony with its surroundings while offering a high degree of performance for physician and patient.

As a general matter relating to stent design, size constraints, fatigue loading modalities, engineering material limitations, and in vivo conditions require that tradeoffs be made in order to balance competing influences so as to offer reliable durability and acceptable outcomes for physician and patient. For example, traditionally a high strength stent may not provide adequate flexibility and/or fatigue resistance. To produce a stent design that offers good physiologic compatibility while offering high strength and fatigue resistance requires a complex balancing of factors in a way that provides for the competing considerations of size, strength, flexibility, durability, and ease of use.

In some embodiments of the present invention there is provided a tubular, mesh-like stent structure comprised of a plurality of interconnected struts. Struts are oriented so as to have a circumferential and an axial direction describing the position of each of the plurality of struts forming the mesh-like tubular structure of the stent.

In many of the embodiments of the present invention, a subset of the plurality struts forming the mesh-like structure are interconnected in a manner that forms a closed structural cell (“cell”), wherein the mesh-like structure is comprised, at least in part, of a plurality of cells which may also be interconnected with a plurality of other adjacent cells.

In some embodiments of the present invention, one or more adjacent cells may be connected by one or more linking struts (“bridges”) as opposed to being directly connected to another adjacent cell.

In some embodiments of the present invention individual struts may have a tapered shape where the strut is widest at its two endpoints and narrowest at a point in between, such as at its midpoint by way of one example. The width profile of the strut taper may be described by two or more segments; each segment being defined by a liner function. The segments may be equal in length or unequal in length and the width of the strut may or may not be symmetric about the midpoint of the strut. Individual struts may have width tapering different from other individual struts in the plurality. Struts may vary in width profiles based on zones along the length of the stent, based on locations within the stent mesh, based on structural variations of individual cells, bridge geometry, and the like.

In some embodiments one or more struts and/or bridges may have curved “s”-like shapes, or other shapes not entirely straight (without regard to a tapered width, if any).

In the most preferred embodiments of the present invention, the point at which two immediately adjacent segments of a strut will have geometric and stress-strain continuity so as to avoid discontinuities that may tend to reduce fatigue performance.

In some embodiments, the stent structure may be comprised of balloon expandable materials known in the art such as alloys of cobalt-chromium, alloys of stainless steel, or any other material that will remain patent and apposed to the luminal wall of a vessel upon the application of a dilating strain from the inflation of a catheter-based balloon.

In some embodiments, the stent structure may be comprised of self-expanding materials known in the art such as nickel-titanium or any other material that will remain patent and apposed to the luminal wall of a vessel at body temperature.

In embodiments where the stent is comprised of nickel-titanium, the material will have a transition temperature where the material is substantially in the austenite phase when unrestrained, the transition temperature being in a range of temperatures at or below nominal body temperature (37 degrees C.), with one preferred range being about 15 degrees C. to about 20 degrees C.

In some embodiments of the present invention the angle of a plurality of struts will be expanded from a closed expansion angle of about zero degrees to an open expansion angle of less than 90 degrees, with the preferred expansion angle being greater than about 45 degrees and less than about 90 degrees.

Described herein is an intraluminal stent having a central longitudinal axis, the intraluminal stent comprising: a closed structural cell having a cell longitudinal axis parallel to the central longitudinal axis and further having a deployment configuration, the closed structural cell comprising: a strut having a length, a first end, and a second end and positioned so as to form at least a portion of a perimeter of the closed structural cell; wherein a width of the strut tapers from the first end and the second end towards a point along the strut length; and wherein when in the deployment configuration, an angle greater than 45 degrees is formed between the cell longitudinal axis and the strut. In some embodiments, the strut has a strut longitudinal axis parallel to the length of the strut, and wherein the width of the strut is measured from an outer surface of the strut to the strut longitudinal axis. In some embodiments, the closed structural cell perimeter is entirely enclosed by the strut and one additional strut. In some embodiments, the closed structural cell perimeter is entirely enclosed by the strut and two additional struts. In some embodiments, the closed structural cell perimeter is entirely enclosed by the strut and three additional struts. In some embodiments, the width of the strut tapers according to a linear relationship between the width and a distance from a point along the length of the strut where the width is narrowest to a point along the strut where the width is widest. In some embodiments, the intraluminal stent comprises an additional closed structural cell that is connected to the structural cell by a linking connection. In some embodiments, a width of the linking connection tapers along at least a portion of a length of the linking connection. In some embodiments, the width of the linking connection tapers according to either a piecewise linear function in relation to the length of the linking connection, a polynomial function in relation to the length of the linking connection, an exponential function in relation to the length of the linking connection, a logarithmic function in relation to the length of the linking connection, or a root function in relation to the length of the linking connection. In some embodiments, the linking connection is S-shaped.

Described herein is an intraluminal stent comprising: a closed structural cell comprising: a strut positioned so as to form at least a portion of a perimeter of the closed structural cell, said strut having a width and a length and comprising a plurality of segments, wherein each segment of the plurality of segments taper according to a linear relationship between the width and the length. In some embodiments, the closed structural cell perimeter is entirely enclosed by the strut and one additional strut. In some embodiments, the closed structural cell perimeter is entirely enclosed by the strut and two additional struts. In some embodiments, the closed structural cell perimeter is entirely enclosed by the strut and three additional struts. In some embodiments, the intraluminal stent comprises an additional closed structural cell directly connected to the closed structural cell by a linking connection. In some embodiments, a width of the linking connection tapers along at least a portion of a length of the linking connection. In some embodiments, the closed structural cell and the additional closed structural cell are offset relative to one another. In some embodiments, the width of the strut is configured so that the strut provides the closed structural cell with a maximum relative flexibility at the narrowest width of the strut and a maximum stiffness at a point of the widest width of the strut. In some embodiments, the closed structural cell is positioned to receive an axial force transmitted by a lumen into which the intraluminal stent is deployed at the point of the widest width of the strut. In some embodiments, the intraluminal stent comprises a nickel titanium alloy, wherein the nickel titanium alloy is substantially in an austenitic phase at a temperature between 15 degrees Celsius to 37 degrees Celsius.

Also described herein is a stent structure for placement in a lumen, the structure comprising: a plurality of interconnected struts forming a tubular mesh-like structure capable of conforming to the inner surface of the lumen, the struts having both a circumferential and axial orientation about the length of the tubular mesh-like structure; a plurality of closed structural cells formed by four struts that describe the perimeter of the cell; a plurality of linking connections between a subset of the structural cells in the tubular mesh-like structure, wherein each strut of a structural cell has a non-uniform width along its length, the width being narrowest at the midpoint of the strut and the width being widest at the two endpoints of the strut; and wherein the non-uniform width of each strut of a structural cell is comprised of two or more zones along its length from endpoint to endpoint, each zone being described by a linear function which determines strut width as a function of lengthwise location on the strut. In some embodiments, one or more of the linking connections are comprised of one or more tapered portions so as to have a portion of the linking connection, located along its length, be narrower than the width at either of its endpoints connecting to struts. In some embodiments, the taper function is selected from one or more of piecewise linear, polynomial, exponential, logarithmic, root. In some embodiments, one or more of the linking connections are s-shaped. In some embodiments, one or more of the linking connections are straight, non-tapered segments. In some embodiments, the unit cell is comprised of interconnected struts forming a diamond shape. In some embodiments, the linear function for a corresponding non-uniform width serves to optimally distribute stress and strain along the length of the strut whose width is defined by the corresponding linear function. In some embodiments, the structure is comprised of a material that obtains its deployed shape through plastic strain deformation. In some embodiments, the material is comprised of a stainless steel alloy. In some embodiments, the material is comprised of a cobalt chromium alloy. In some embodiments, the structure is comprised of a material that obtains its deployed shape through self-expansion. In some embodiments, the material is comprised of nickel titanium. In some embodiments, the nickel-titanium material is substantially in the austenitic phase at a given temperature ranging from 15 degrees Celsius to 37 degrees Celcius. In some embodiments, an angle of expansion formed by adjacent and connected struts of a unit cell ranges from 0 degrees to 90 degrees. In some embodiments, the mesh structure comprised of unit cells is comprised of a symmetric mesh pattern along the length of the stent structure. In some embodiments, the mesh structure comprised of unit cells is comprised of an asymmetric mesh pattern along the length of the stent structure. In some embodiments, the asymmetric mesh pattern is repeated in sections along the length of the stent structure. In some embodiments, a plurality of asymmetric mesh patterns exists along the length of the stent structure. In some embodiments, the struts comprising the mesh have a tapered shape and spatial arrangement such that the edges of adjacent struts nest together when at a constrained diameter so as to provide a reduced delivery catheter profile. In some embodiments, adjacent unit cells are positionally offset within the mesh structure to provide further nesting.

### BRIEF DESCRIPTION OF THE FIGURES

FIGS. 1A-1B, 2A-2B, 3A-3B, and 4A-4B are schematic representations of exemplary stent embodiments showing example strut taper shapes and closed-cell structures formed by individual struts.

FIGS. 5A-5C are schematic representations of exemplary stent embodiments showing an example of a stent repeating structural unit having a closed cell configuration with interconnection between two or more cells.

FIGS. 6A-6C are schematic representations of exemplary stent embodiments showing an example of a stent repeating structural unit having a closed cell configuration with interconnection between two or more cells.

FIGS. 7A-7C are schematic representations of exemplary stent embodiments showing an example of a stent repeating structural unit having a closed cell configuration with interconnection between two or more cells.

FIGS. 8A and 8B show force diagrams for strut expansion angles.

FIG. 9 is a graph showing the increase in comparative stiffness levels of examples of stent embodiments as a function of diametral compression in comparison to a widely used prior art stent.

FIG. 10 is a graph showing the comparative strain levels of exemplary stent embodiments as a function of diametral compression in comparison to a widely used prior art stent.

### DETAILED DESCRIPTION

In some embodiments of the present invention, individual struts may have a tapered shape where the strut is widest at its two endpoints and narrowest at a point in between, such as at its midpoint by way of one example. Tapering, in some embodiments is in accordance with any mathematical function, including polynomial functions, exponential functions, logarithmic functions, root functions, and any combination thereof. The width profile of the strut taper may be described by a plurality of segments; each segment being defined by a function along each segment. The segments may be equal in length or unequal in length and the width of the strut may or may not be symmetric about the midpoint of the strut. Individual struts may have width tapering different from other individual struts in the plurality. Struts may vary in width profiles based on zones along the length of the stent, based on locations within the stent mesh, based on structural variations of individual cells, bridge geometry, and the like. In several embodiments of the present invention, the plurality of segments are described by linear functions and the strut segments are comprised of piecewise linear tapering segments.

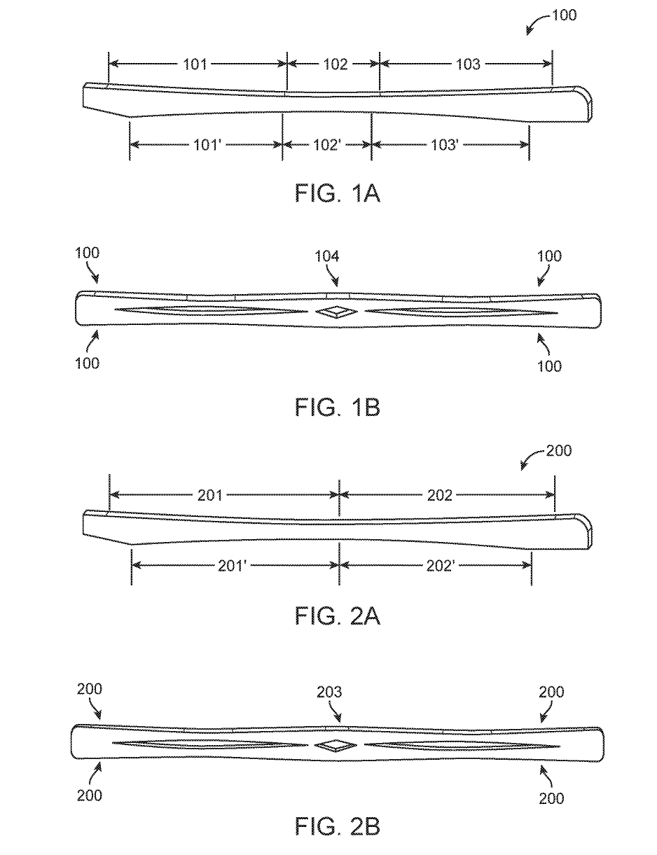

Referring to FIGS. 1A and 1B, an example strut shape 100 has an external edge comprised of sections 101, 102, 103 and an internal edge comprised of sections 101′, 102′, 103′. The edge of each section may be defined by a liner function (w)=f(l), where “w” is strut width and “l” is strut length, that describes the overall taper of the strut 100 from its maximum widths at its end points toward a minimum width there between. One convenient way to define the linear relationship of strut width as a function of strut length over an individual edge section, is to calculate width from an imaginary line running along an approximately central axis through the length of the body of the strut. In the present example, moving from left to right in FIG. 1A, outer surface 101 and inner surface 101′ taper linearly as they respectively approach edge sections 102 and 102′. In the present example, edge sections 102 and 102′ are constant width, however, one or each of 102 and 102′ may themselves describe a linear taper and may further be asymmetrical. Where 102 intersects 103, and where 102′ intersects 103′, the outer and inner strut edges respectively increase linearly in width to the right endpoint of strut 100.

Closed cell 104 is shown as one example of how a plurality of individual struts may be used to create a structural unit. In the example of FIG. 1B, closed cell 104 is comprised of four struts 100, however, a closed cell structural design may be comprised of two or more struts, and, each strut may be comprised of its own shape depending on the desired mechanical characteristics of the closed cell structure. For example, a three-sided closed cell may have one, two, or three different strut shapes respectively comprising each of the sides of the closed cell, and so forth. A preferred embodiment of the present invention uses symmetrical cell construction in order to accommodate the complex loading conditions of the venous system in the leg, however, for a location with lesser or more static loading conditions, asymmetrical designs may be tailored to fit the application in question.

Referring now to FIGS. 2A and 2B, an example strut shape 200 has an external edge comprised of sections 201, 202 and an internal edge comprised of sections 201′, 202′. The edge of each section may be defined by a liner function (w)=f(l), where “w” is strut width and “l” is strut length, that describes the overall taper of the strut 200 from its maximum widths at its end points toward a minimum width there between. In the present example, moving from left to right in FIG. 2A, outer surface 201 and inner surface 201′ taper linearly as they respectively approach edge sections 202 and 202′, and optionally may themselves asymmetrical. Where 202 intersects 201, and where 202′ intersects 202′, the outer and inner strut edges respectively increase linearly in width to the right endpoint of strut 200, and optionally may themselves asymmetrical.

In the example of FIG. 2B, closed cell 203 is comprised of four struts 200, and as previously described, may optionally be comprised of three or more struts having a symmetrical or an asymmetrical construction.

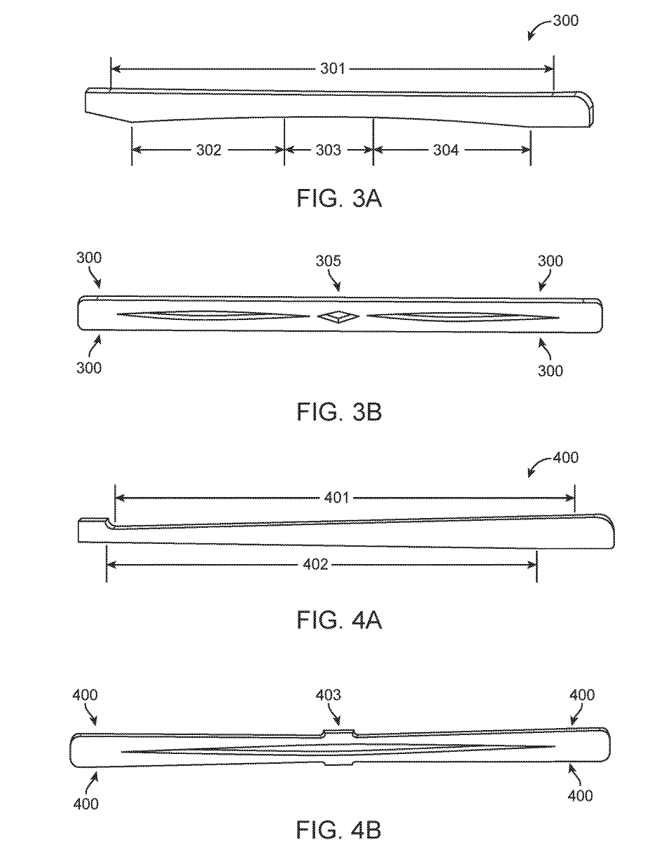

Referring now to FIGS. 3A and 3B, an example strut shape 300 has an external edge comprised of section 301 and an internal edge comprised of sections 302, 303, 304. The edge of each section may be defined by a liner function (w)=f(l), where “w” is strut width and “l” is strut length, that describes the overall taper of the strut 300 from its maximum widths at its end points toward a minimum width there between. In the present example, moving from left to right in FIG. 3A, outer surface 301 is asymmetrical from the inner edge. As shown, outer edge section 301 is a constant linear function, however 301 may optionally increase or decrease in width moving from left to right. Inner edge section 302 tapers in width as it approaches edge section 303. Inner edge section 303 is shown as a constant linear function, however it optionally may increase or decrease in width as it approaches edge section 304. Inner edge section 304 increases linearly in width as it approaches the right endpoint of strut 300.

In the example of FIG. 3B, closed cell 305 is comprised of four struts 300, and as previously described, may optionally be comprised of three or more struts having a symmetrical or an asymmetrical construction.

Referring now to FIGS. 4A and 4B, an example strut shape 400 has an external edge comprised of section 401 and an internal edge comprised of section 402. The edge of each section may be defined by a liner function (w)=f(l), where “w” is strut width and “l” is strut length, that describes the overall taper of the strut 400 from its minimum width at one end point toward a maximum width at its opposite end point. In the present example, moving from left to right in FIG. 4A, outer surface 401 is a linear function increasing in width, and inner edge section 402 increases in width. Edge sections 401 and 402 may optionally increase in width at different linear rates.

In the example of FIG. 4B, closed cell 403 is comprised of four struts 400, and as previously described, may optionally be comprised of three or more struts having a symmetrical or an asymmetrical construction.

In many of the embodiments of the present invention, a subset of the plurality struts forming the mesh-like structure are interconnected in a manner that forms a closed structural cell (“cell”), wherein the mesh-like structure is comprised, at least in part, of a plurality of cells which may also be interconnected with a plurality of other adjacent cells. In this fashion, one or more types of structural cell units may be linked or directly joined together to form the complete mesh of a stent. The mesh structure may have structural symmetry or asymmetry depending on the desired mechanical characteristics of the stent, e.g. fatigue resistance, axial or radial stiffness, torsional stiffness, flexibility in bending, and the like.

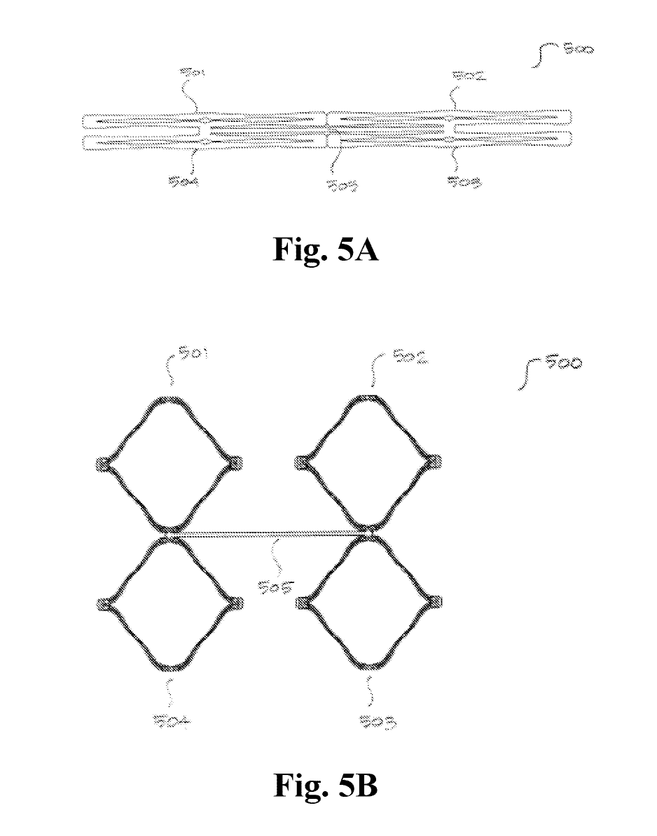

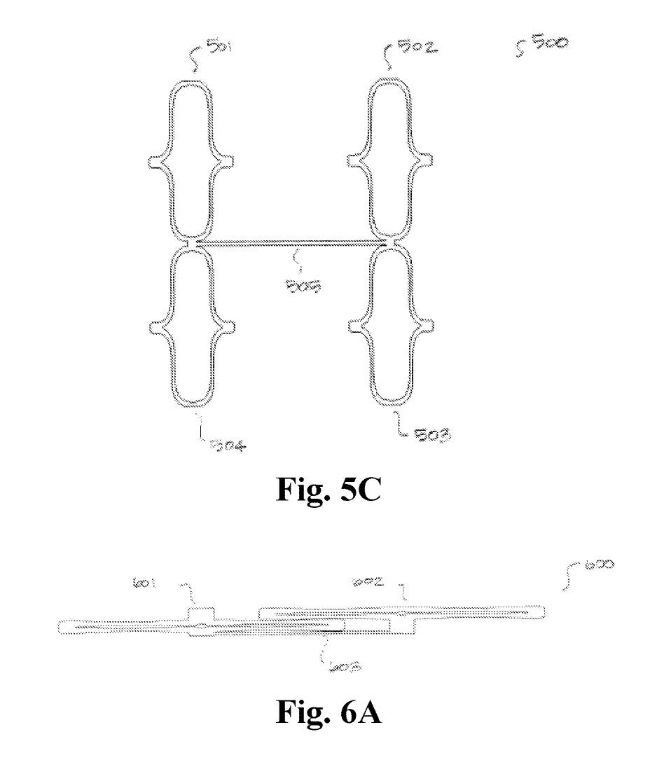

Referring now to FIGS. 5A-7C, one or more adjacent cells may be connected, which may further include one or more bridges as opposed to being directly connected to another adjacent cell. Optionally, struts and/or bridges may have curved “s”-like shapes, or other shapes not entirely straight (without regard to a tapered width, if any). To achieve maximum fatigue resistance, it is preferred that the point at which two immediately adjacent segments of a strut has geometric and stress-strain continuity so as to avoid discontinuities that may negatively affect fatigue performance.

In FIG. 5A, a plurality of unit cells 501, 502, 503, 504, and a bridge 505 comprise a larger structural unit 500 that is shown in its unexpanded state. Each of the unit cells may be comprised of struts which themselves are symmetric or asymmetric, and each strut of the individual unit cells may be alike or individually shaped depending on the desired mechanical performance. In a preferred embodiment, both the individual struts comprising unit cells 501, 502, 503, 504 are symmetric, and the unit cells comprising structure 500 are also symmetric.

In the example of FIG. 5B, structure 500 is partially expanded where it is visible how the structure 500 is comprised to include bridge 505 which may serve to connect one grouping of closed cells to another grouping of closed cells. The shape, length, and width of bridge 505 may be optimized to achieve the desired axial stiffness, bending stiffness, and fatigue resistance of the macroscopic stent mesh design of which structure 500 may be a part. Bridge 505 may optionally connect groupings of unit cells at differing relative locations. For example, bridge 505 is shown to connect at the central intersection of between the grouping 501, 504 and the grouping 502, 503. Bridge 505 could also connect between the bottom of cell 504 and the top of cell 502, or the bottom of cell 504 and the top of cell 503, or the top of cell 501 and the bottom of cell 503, etc.

FIG. 5C shows structure 500 with the struts of unit cells 501, 502, 503, 504 expanded to their maximum angle of expansion from the unexpanded or collapsed configuration shown in FIG. 5A.

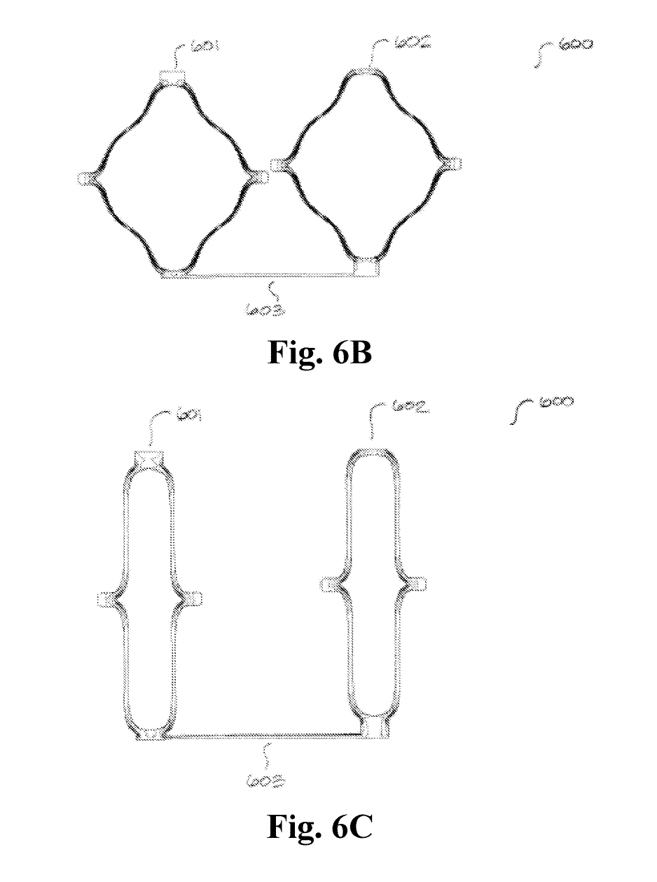

In FIG. 6A, a plurality of unit cells 601, 602 and a bridge 603 comprise a larger structural unit 600 that is shown in its unexpanded state. Each of the unit cells may be comprised of struts which themselves are symmetric or asymmetric, and each strut of the individual unit cells may be alike or individually shaped depending on the desired mechanical performance. In a preferred embodiment, both the individual struts comprising unit cells 601, 602 are symmetric, and the unit cells comprising structure 600 are also symmetric.

In the example of FIG. 6B, structure 600 is partially expanded where it is visible how the structure 600 is comprised to include bridge 603 which may serve to connect one grouping of closed cells to another grouping of closed cells. The shape, length, and width of bridge 603 may be optimized to achieve the desired axial stiffness, bending stiffness, and fatigue resistance of the macroscopic stent mesh design of which structure 600 may be a part. Bridge 603 may optionally connect groupings of unit cells at differing relative locations. For example, bridge 603 is shown to connect at the base of cells 601 and 602. Bridge 603 could also connect between the tops of cells 601 and 602, etc.

FIG. 6C shows structure 600 with the struts of unit cells 601, 602 expanded to their maximum angle of expansion from the unexpanded or collapsed configuration shown in FIG. 6A.

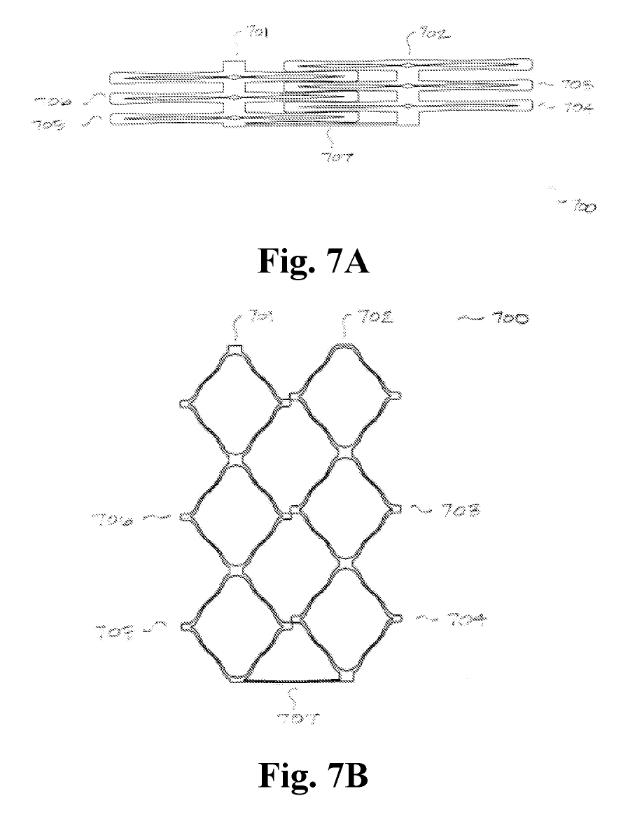

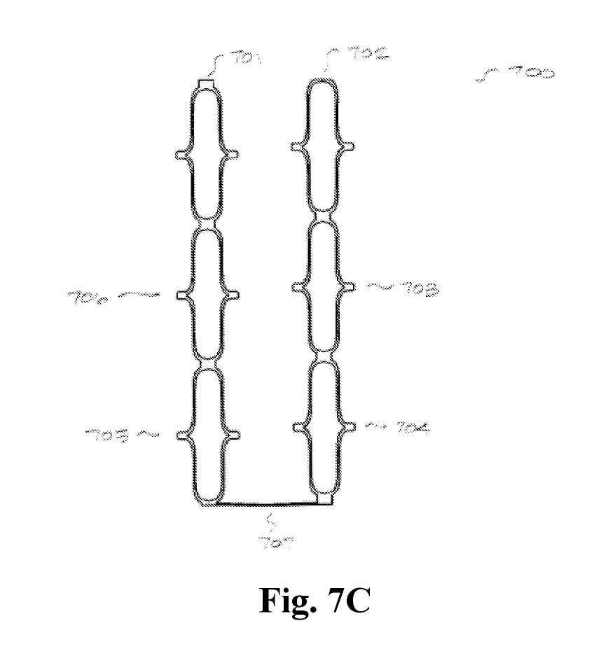

In FIG. 7A, a plurality of unit cells 701, 702, 703, 704, 705, 706 and a bridge 707 comprise a larger structural unit 700 that is shown in its unexpanded state. Each of the unit cells may be comprised of struts which themselves are symmetric or asymmetric, and each strut of the individual unit cells may be alike or individually shaped depending on the desired mechanical performance. In a preferred embodiment, both the individual struts comprising unit cells 701, 702, 703, 704, 705, 706 are symmetric, and the unit cells comprising structure 700 are also symmetric.

In the example of FIG. 7B, structure 700 is partially expanded where it is visible how the structure 700 is comprised to include bridge 707 which may serve to connect one grouping of closed cells to another grouping of closed cells. The shape, length, and width of bridge 707 may be optimized to achieve the desired axial stiffness, bending stiffness, and fatigue resistance of the macroscopic stent mesh design of which structure 700 may be a part. Bridge 707 may optionally connect groupings of unit cells at differing relative locations. For example, bridge 707 is shown to connect at the base of cells 705 and 704. Bridge 707 could also connect between the tops of cells 701 and 702, or between the adjacent ends of 705 and 704, or between the adjacent ends of 706 and 703, etc.

FIG. 7C shows structure 700 with the struts of unit cells 701, 702, 703, 704, 705, 706 expanded to their maximum angle of expansion from the unexpanded or collapsed configuration shown in FIG. 7A.

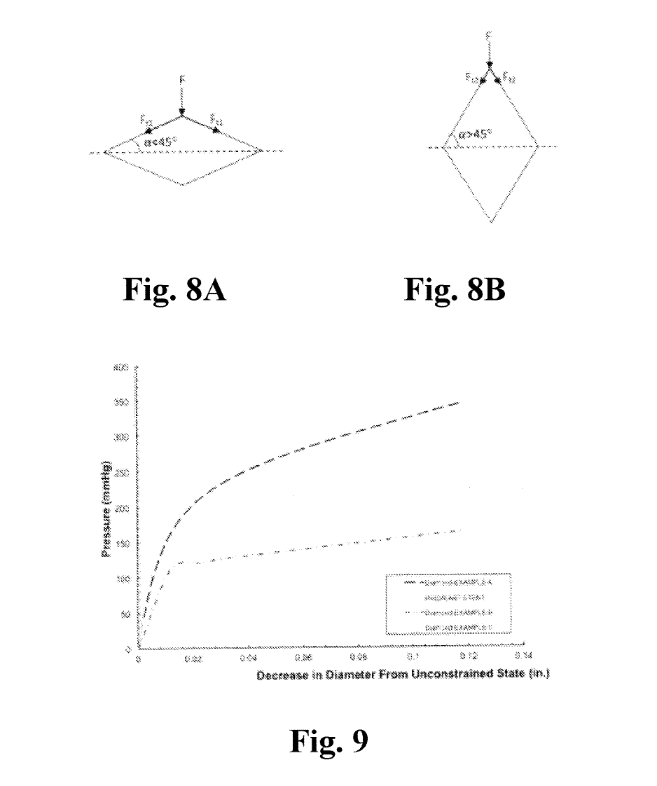

Referring now to FIGS. 8A and 8B, in some embodiments of the present invention the angle of a plurality of struts will be expanded from a closed expansion angle of about zero degrees to an open expansion angle of less than 90 degrees, with the preferred expansion angle being greater than about 45 degrees and less than about 90 degrees. As seen in comparison between the cells of FIG. 8A and FIG. 8B, a unit cell comprised of equal struts is shown where expansion angle α is less than about 45 degrees. If a force F is applied at the apex of two connected struts, the force F will apply a transmitted load along the two struts, forces Fₜ₁ and Fₜ₂ respectively. The moment arm by which forces Fₜ₁ and Fₜ₂ act to cause the struts to collapse from their expanded angles is the cosine of angle α. As may be observed in FIG. 8A, the smaller the angle α, the larger its cosine, and hence, the longer the moment arm upon which forces Fₜ₁ and Fₜ₂ act to collapse the expanded struts. Conversely, as may be observed in FIG. 8B, the larger the angle α, the smaller its cosine, and hence, the shorter the moment arm upon which forces Fₜ₁ and Fₜ₂ act to collapse the expanded struts. The net result of this principle of basic mechanics is that a comparatively high degree of radial stiffness for a stent design may, in part, be accomplished by the degree to which struts are expanded. As is also understood from basic mechanics, as higher angles of expansion a are used, the columnar buckling strength of a strut becomes a consideration in the overall design of a stent mesh.

In some embodiments of the present invention there is provided a tubular, mesh-like stent structure comprised of a plurality of interconnected struts. Struts are oriented so as to have a circumferential and an axial direction describing the position of each of the plurality of struts forming the mesh-like tubular structure of the stent. The orientation of struts, individual strut geometries, the thickness of struts (distance between the surfaces forming the inner diameter and outer diameter of the mesh from a center point of its diameter), the geometry of the intersections between struts and unit cells, bridge geometry, materials of composition, and thermomechanical properties such as material state of phase and residual strain all form a complex set of factors that will influence both the design and the optimized performance of a stent mesh for a particular application.

In some embodiments, the stent structure may be comprised of balloon expandable materials known in the art such as alloys of cobalt-chromium, alloys of stainless steel, or any other material that will remain patent and apposed to the luminal wall of a vessel upon the application of a dilating strain from the inflation of a catheter-based balloon.

In some embodiments, the stent structure may be comprised of self-expanding materials known in the art such as nickel-titanium or any other material that exhibits shape memory—superelastic behavior capable of remaining patent and apposed to the luminal wall of a vessel at body temperature.

In embodiments where the stent is comprised of nickel-titanium, the material will have a transition temperature where the material is substantially in the austenite phase when unrestrained, the transition temperature being in a range of temperatures at or below nominal body temperature (37 degrees C.), with one preferred range being about 0 degrees C. to about 30 degrees C.

The embodiments of the present invention are particularly advantageous for use in catheter-based systems and medical procedures where the desire to provide minimally invasive procedures to deliver robust implantable structures to treat complex conditions of the body is coupled with patient quality of life based on clinical outcomes and the unmet needs of providing implants with high strength and fatigue resistance.

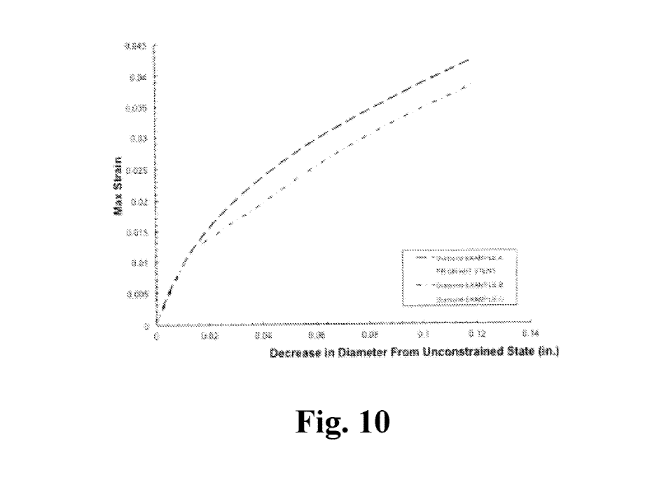

Referring now to FIGS. 9-10 for comparisons of radial pressure as example embodiments of the present invention (in FIGS. 5A-7C) as compared to a very widely used prior art stent (insertion of FIG. 9-10). In FIG. 9, “EXAMPLE A” corresponds to a stent mesh comprised of structure 500, “EXAMPLE B” corresponds to a stent mesh comprised of structure 600, and “EXAMPLE C” corresponds to a stent mesh comprised of structure 700. As can be seen from each of the EXAMPLE plots, there is a greater radial pressure for the same reductions in diameter up to about 0.035 inches as compared to the reference prior art stent. Additionally, EXAMPLE A provides greater stiffness for diameter reductions of about 0.07 inches as compared to the reference prior art stent. Hertzberg, et al. (A J R Am. J. Roentgenol., 1997 May; 168(5):1253-7) (the entire contents of which are incorporated herein by reference), reported that average leg veins range in diameter from about 10 mm to about 7 mm, with diameters decreasing when traveling to the extremity of the leg. By observation of the plots of FIG. 9 and applying the reported average size of leg veins, it may be conservatively concluded that example embodiments of the present invention provide grater radial stiffness for diameter reductions of about 9% or more, in the leg, depending on the mesh structure.

In FIG. 10, the EXAMPLE plots correspond to the same mesh structures of the present invention as described by FIG. 9. In FIG. 10, strain is plotted as a function of decrease in diameter. Each EXAMPLE plot shows reduced strain levels as a function of diameter reduction. The tapering of the struts in accordance with the present invention provides improved distribution of stress and strain when compared to a uniform strut width. Moreover, the tapered strut shape may provide for nesting of adjacent strut structures when at a constrained diameter so as to provide for a reduced delivery catheter profile as compared to structures of uniform strut widths while maintaining a desirable combination of radial stiffness, radial strength, and flexibility.

As has been described herein, treatment of venous stenosis is better served by having a stent design that directly addresses the unique requirements and differences in the vein as opposed to using a stent designed and optimized for arterial use where loading conditions and tissue behavior are different than the vein. The higher radial strength offered by the present invention, in combination with the improvement in load distribution offered by the linear tapering of strut shapes of the present invention offers a combination of advantages. Nonetheless, embodiments of the present invention may be used in arterial, cardiac, and other physiologic applications where patency of a lumen and fatigue resistance are useful.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims. Other embodiments are within the following claims.

What is claimed is:

1. An intraluminal stent having a central longitudinal axis, the intraluminal stent comprising:
a closed structural cell having a cell longitudinal axis parallel to the central longitudinal axis and further having a deployment configuration, the closed structural cell comprising:
a strut having a length, a first end, and a second end and positioned so as to form at least a portion of a perimeter of the closed structural cell;
wherein a width of the strut tapers from the first end and the second end towards a point along the strut length; and
wherein when in the deployment configuration, an angle greater than 45 degrees is formed between the cell longitudinal axis and the strut.

2. The intraluminal stent of claim 1, wherein the strut has a strut longitudinal axis parallel to the length of the strut, and wherein the width of the strut is measured from an outer surface of the strut to the strut longitudinal axis.

3. The intraluminal stent of claim 1, wherein the closed structural cell perimeter is entirely enclosed by the strut and one additional strut.

4. The intraluminal stent of claim 1, wherein the closed structural cell perimeter is entirely enclosed by the strut and two additional struts.

5. The intraluminal stent of claim 1, wherein the closed structural cell perimeter is entirely enclosed by the strut and three additional struts.

6. The intraluminal stent of claim 1, wherein the width of the strut tapers according to a linear relationship between the width and a distance from a point along the length of the strut where the width is narrowest to a point along the strut where the width is widest.

7. The intraluminal stent of claim 1, comprising an additional closed structural cell that is connected to the structural cell by a linking connection.

8. The intraluminal stent of claim 7, wherein a width of the linking connection tapers along at least a portion of a length of the linking connection.

9. The intraluminal stent of claim 8, wherein the width of the linking connection tapers according to either a piecewise linear function in relation to the length of the linking connection, a polynomial function in relation to the length of the linking connection, an exponential function in relation to the length of the linking connection, a logarithmic function in relation to the length of the linking connection, or a root function in relation to the length of the linking connection.

10. The intraluminal stent of claim 7, wherein the linking connection is S-shaped.

11. An intraluminal stent comprising:
a closed structural cell comprising:
a strut positioned so as to form at least a portion of a perimeter of the closed structural cell, said strut having a width and a length and comprising a plurality of segments, wherein each segment of the plurality of segments taper according to a linear relationship between the width and the length.

12. The intraluminal stent of claim 11, wherein the closed structural cell perimeter is entirely enclosed by the strut and one additional strut.

13. The intraluminal stent of claim 11, wherein the closed structural cell perimeter is entirely enclosed by the strut and two additional struts.

14. The intraluminal stent of claim 11, wherein the closed structural cell perimeter is entirely enclosed by the strut and three additional struts.

15. The intraluminal stent of claim 11, comprising an additional closed structural cell directly connected to the closed structural cell by a linking connection.

16. The intraluminal stent of claim 15, wherein a width of the linking connection tapers along at least a portion of a length of the linking connection.

17. The intraluminal stent of claim 15, wherein the closed structural cell and the additional closed structural cell are offset relative to one another.

18. The intraluminal stent of claim 11, wherein the width of the strut is configured so that the strut provides the closed structural cell with a maximum relative flexibility at the narrowest width of the strut and a maximum stiffness at a point of the widest width of the strut.

19. The intraluminal stent of claim 11, wherein the closed structural cell is positioned to receive an axial force transmitted by a lumen into which the intraluminal stent is deployed at the point of the widest width of the strut.

20. The intraluminal stent of claim 11, comprising a nickel titanium alloy, wherein the nickel titanium alloy is substantially in an austenitic phase at a temperature between 15 degrees Celsius to 37 degrees Celsius.
